(19) United States
(12) Patent Application Publication
(10) Pub. No.: US 2003/0055507 A1
(43) Pub. Date: Mar. 20, 2003

(54) Modular prosthesis and insertion tool for bone structures

(75) Inventors: Dennis McDevitt, Raleigh, NC (US); George Hadley Callaway, Raleigh, NC (US)

Correspondence Address: RYAN KROMHOLZ & MANION, S.C., Post Office Box 26618, Milwaukee, WI, 53226-0618, US

(73) Assignee: Incumed, Incorporated

(21) Appl. No.: 10/034,343

(22) Filed: Dec. 28, 2001

Related U.S. Application Data
(60) Provisional application No. 60/322,170, filed on Sep. 15, 2001.

(51) Int. Cl. A61F 2/40

(52) U.S. Cl. 623/19.11; 623/23.48

(57) ABSTRACT

A prosthesis is attached to bone by means of a stem that expands in a bone structure in response to the insertion of a pin.

### RELATED APPLICATION

This application claims the benefit of the filing date of copending United States Provisional Patent Application Serial No. 60/322,170, filed Sep. 11, 2001, entitled “Modular Prosthesis for Bone Structures.”

### FIELD OF THE INVENTION

The present invention relates to prosthetic implants and, in particular, to an implant for a glenoid cavity of a shoulder joint.

### BACKGROUND OF THE INVENTION

A shoulder joint consists of a ball-and-socket type coupling of the humerus to the scapula. The humerus forms the ball, and the socket is formed at the glenoid cavity of the scapula. Injury or disease may cause the destruction or deterioration of the glenoid cavity, making normal functioning and use of the joint painful or not possible. In this situation, a replacement joint surface for the glenoid cavity may be desired.

Modular prosthetic implants of the glenoid cavity of a shoulder joint, which are targeted at repairing the glenoid cavity of the scapula, are known. For example, multiple section glenoid implants that incorporate a single or multiple stem structures on a medial side of the prosthesis to aid fixation to a scapula bone have been used. Stemmed implants typically require considerable resection (removal of bone) of the scapula to insure a proper fit. Further, stemmed structures require bone cement, bone screws or their combination to secure the implant to the scapula.

### SUMMARY OF THE INVENTION

The invention provides a prosthesis that can be used in any joint requiring an implant. The prosthesis can be secured to bone without use of additional fasteners or bone cement. The prosthesis also makes possible an implant that requires less resection of bone.

One aspect of the invention provides a prosthetic implant for a bone structure. The implant comprises a base that includes at least one stem adapted to engage at least one void created in the bone structure. The stem has an exterior peripherally surrounding an interior lumen. The implant also includes a pin that fits into the interior lumen and, when fitted, expands the exterior of the stem. Expansion of the stem compresses surrounding bone structure, to thereby secure the base to the bone structure.

In one embodiment, the implant further includes a cap from which the pin depends. Fitting the pin into the interior lumen of the stem couples the cap to the base. The cap can include a bearing surface. In this arrangement, the pin depends from the cap from a surface that faces away from the bearing surface. The cap and base can also include nesting surfaces that rest together when the base is coupled to the cap.

Another aspect of the invention provides a method of mounting a prosthesis in a bone structure. The method provides an implant that includes a base having at least one stem having an exterior peripherally surrounding an interior lumen, and a pin that fits into the interior lumen. The method locates the implant on the bone structure by placing the stem into a void formed in the bone structure. The method secures the implant by inserting the pin in the interior lumen of the stem to expand the exterior of the stem within surrounding bone structure. The method is useful, e.g., for installing an implant in a glenoid cavity of a shoulder joint.

Another aspect of the invention provides systems and methods for installing a prosthetic implant for the glenoid cavity of a shoulder joint.

In one embodiment, the invention provides a prosthetic implant for the glenoid cavity of a shoulder joint. The implant can comprise, for example, a base and a cap.

In this arrangement, the base has, for example, three stems protruding in a generally perpendicular manner from an inferior surface of the base. The stems are located in such a manner as to engage prepared cavities in the bone, for example, three reamed holes in a scapula. The stems fit into the prepared cavities with a secure fit. Each stem has side exposed to bone. The exposed side is, for example, textured to enhance fixation to new bone growth. Each stem also has a hollow interior and open top. The hollow interior and open top are adapted to receive a pin. A superior surface of the base has openings corresponding with the open top and hollow interior of the stems. There is, for example, a slight taper at the openings to facilitate engagement of the pin.

In one embodiment, the cap of the modular prosthesis has a bearing surface and an opposite facing mounting surface. The bearing surface of the cap is adapted to engage a bone or separate implant and is generally contoured to receive such implant or bone. The mounting surface has, for example, an indentation adapted to receive the superior surface of the base, whereby the superior surface of the base is nested in the indentation of the cap.

Also protruding in a generally perpendicular fashion, and being rigidly attached to the cap, is at least one pin. The pin is adapted to be received by a corresponding opening on the base. When the pin is inserted in the opening of the base, and is fully engaged with the open top and hollow interior of the stem, an interference fit is created in the bone, because the pin forces the stem to expand inside the void of the bone. This expansion creates a compressive mechanical lock of the prosthesis in the bone.

The use of an expanding pin that is integral to the prosthesis for fastening the prosthesis to the bone eliminates the need for additional fasteners or bone cement. However, if desired, additional fastening means can be used.

The material of the cap and base need not be identical. Materials commonly used in the prosthetic arts can be used, including, but not limited to, metals, ceramics, titanium, titanium alloys, tantalum, chrome cobalt, surgical steel

### DESCRIPTION OF THE DRAWINGS

FIG. 1 is an anterior and exploded view of the shoulder region of a human skeleton.

FIG. 2 is an exploded perspective showing the present invention highlighting the bearing surface.

FIG. 3 is a perspective and exploded view of the present invention highlighting the pins and stems.

FIG. 4 is an exploded view showing the assembly of the modular prosthesis in relation to the shoulder region of a human skeleton.

FIG. 5 is a detailed cross sectional view depicting the stem inserted in a bone.

FIG. 6 is a detailed cross sectional view depicting the assembled modular prosthesis in the scapula, highlighting the expanded pins of the top portion engaged with the stem of the bottom portion of the prosthesis of this invention.

FIG. 7 is an exploded view of an insertion assembly embodying features of the present invention.

FIG. 8 is an assembled view of the assembly shown in FIG. 7.

FIG. 9 is a perspective view illustrating the use of an insertion tool to place the cap component onto the base component implanted within the glenoid cavity.

FIG. 10 illustrates the use of a hammer to mount the cap onto the base.

FIG. 11 illustrates the manipulation of the insertion tool to release the cap component.

FIG. 12 illustrates the removal of the insertion tool from the glenoid cavity and further illustrating the cap and base components of the modular prosthesis implanted within the glenoid cavity.

Other objects, advantages, and embodiments of the invention are set forth in part in the description which follows, and in part, will be obvious from this description, or may be learned from the practice of the invention.

### DETAILED DESCRIPTION OF THE PREFERRED EMBODIMENTS

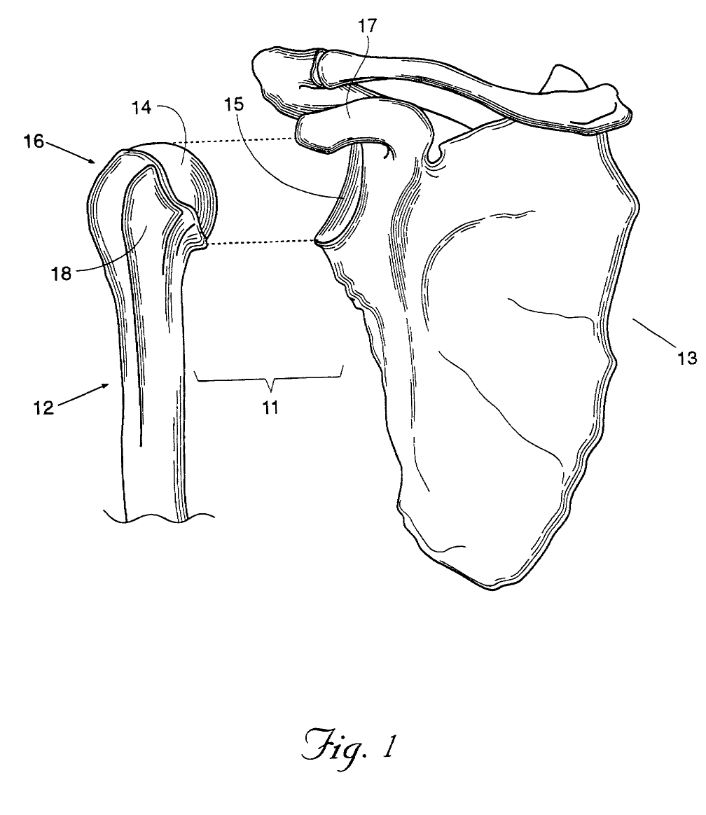

FIG. 1 shows a shoulder skeleton in an exploded view. An upper portion of an arm bone or humerus 12 is shown. The humerus has a ball 14 that is rotatably received by a glenoid cavity 15. The ball 14 is adjacent to a greater tubercle 16 and a lesser tubercle 18.

Opposite the humerus 12 is the shoulder blade or scapula 13. The intersection of the scapula 13 and the humerus 12 is known as the shoulder joint 11. The scapula 13 has a coracoid process 17 protruding toward the humerus 12. As mentioned, the humerus 12 rotatably engages the scapula 13 at the glenoid cavity 15. Working with the ball 14, the glenoid cavity 15 is the socket for the shoulder joint 11, which is referred to as a ball and socket joint.

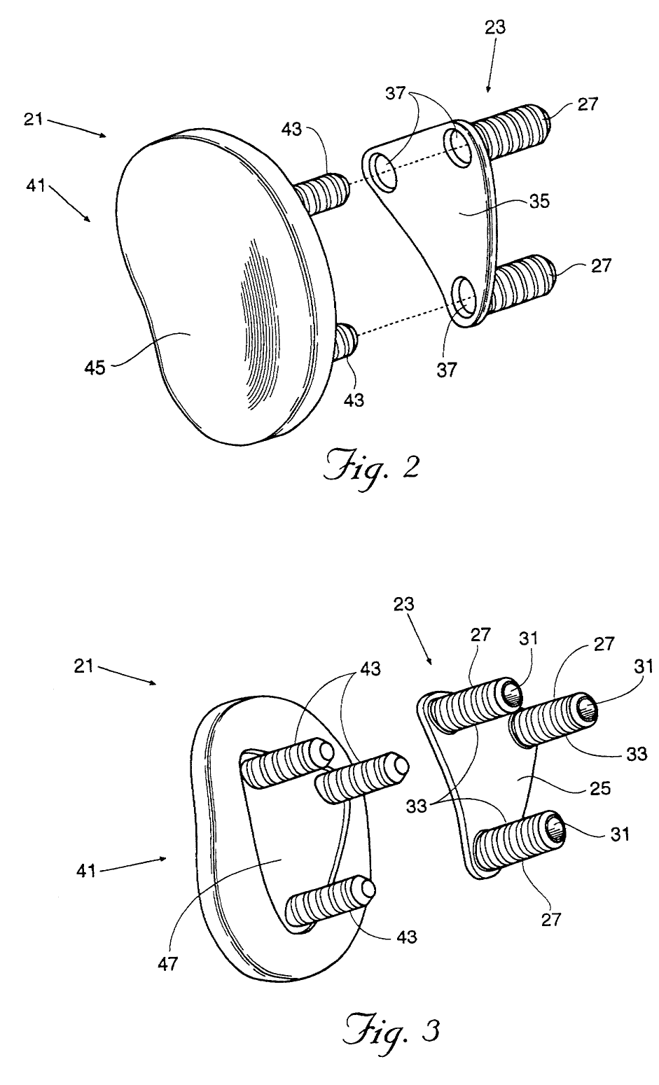

FIGS. 2 and 3 show one embodiment of the present invention. In this embodiment, a modular prosthesis 21 comprises a base 23 and a cap 41. The base 23 has three stems 27 which, in use, engage the glenoid cavity at defined holes adapted to receive the stems 27, as will be described later. As FIGS. 2 and 3 show, the stems 27 are desirably ribbed or threaded.

Each stem 27 has a hollow interior 31 and an exposed side 33. Each stem 27 intersects a superior surface 35 and passes through an inferior surface 25 of the base 23. The orientation of the stems 27 with respect to each other is not critical. A factor, for example, used to determine the orientation of each stem 27 would be the particular geometry of the glenoid cavity of a selected patient.

The superior surface 35 is adapted to interface with the cap 41. Moreover, the stems 27 and the respective hollow interior 31 are adapted to receive corresponding pins 43 carried by the cap 41. The pins 43 are sized to create an interference fit within the stems 27, once seated in the corresponding hollow interior 31 of the respective stem 27. The insertion of the pins 43 into the stems 27 causes a compression of the surrounding bone matter of the scapula. As FIGS. 2 and 3 show, the pins 43 are desirably ribbed or threaded.

The cap 41 also has a bearing surface 45 which is adapted to rotatably receive the ball 14 of the humerus 12, as FIG. 1 shows.

In one embodiment, to ensure proper seating of the cap 41 in the base 23, and to ensure proper orientation of the bearing surface 45, the base 23 has a superior surface 35 adapted to seat with the mounting surface 47 of the cap 41.

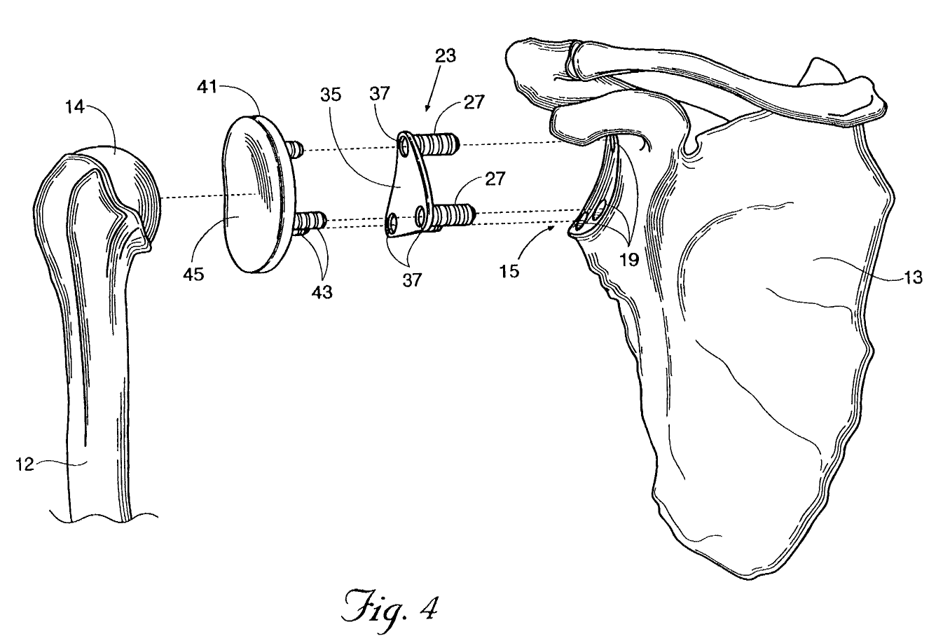

FIG. 4 depicts an embodiment of the modular prosthesis fitted to bone. The scapula 13 has been adapted to receive the prosthesis at the glenoid cavity 15. The glenoid cavity 15 may, for example, be sculpted or resected by a surgeon to ensure proper alignment of the base section 23 of the prosthesis. By any suitable means known in the relevant art, corresponding mounting holes 19 are drilled, reamed or otherwise shaped into the glenoid cavity 15. Each hole 19 is oriented to receive a stem 27 of the base 23. As FIG. 4 shows, the base 23 is oriented such that the single (apex) stem 27 of the triangular array of stems 27 occupies the top (superior) position. The cap 41 then fits over the base section 23. Pins 43 on the cap 41 align with corresponding hollow 31 stems 27 of the base, while the mounting surface of the cap 43 seats on the superior surface 35 of the base 23.

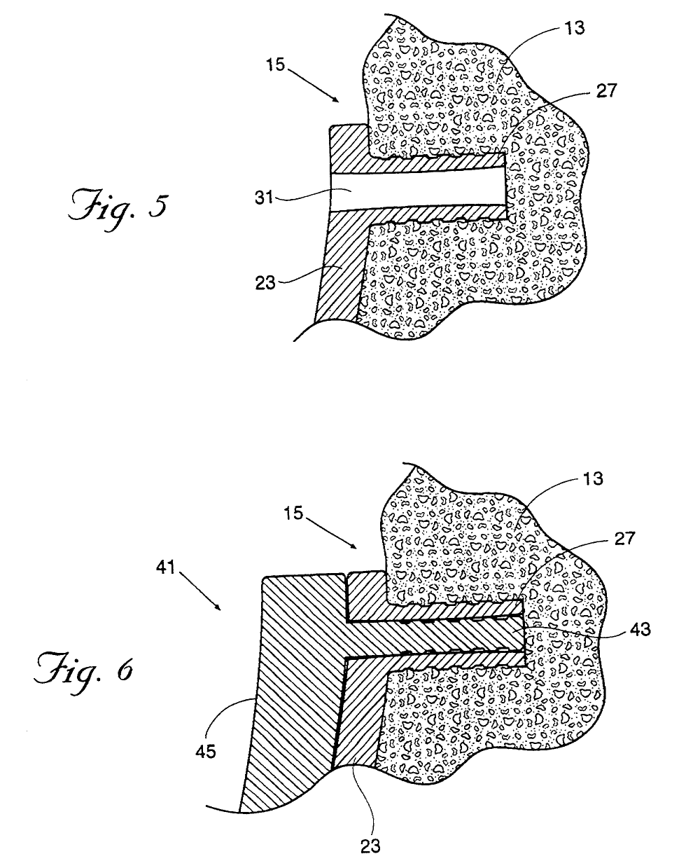

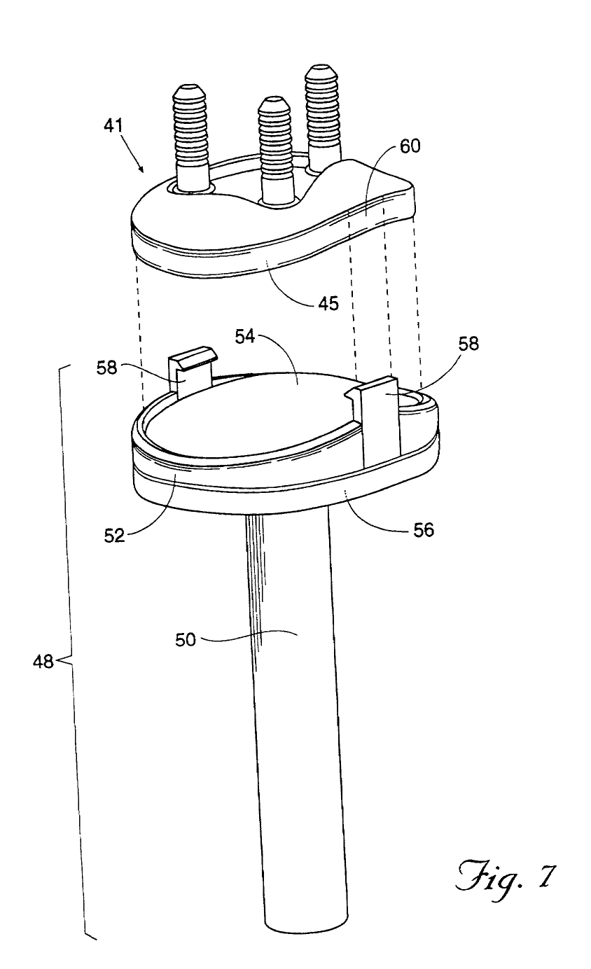

As FIGS. 5 and 6 show, as the pins 43 engage the openings 37 surrounding the hollow inside 31 of each stem 27, the stem 27 swells and thereby places the surrounding bone matter of the scapula 13 in compressive opposition to the stem 27, thus creating a coupling of the prosthesis to the scapula 13. The humerus 12 then rests against the bearing surface 45 of the cap 41.

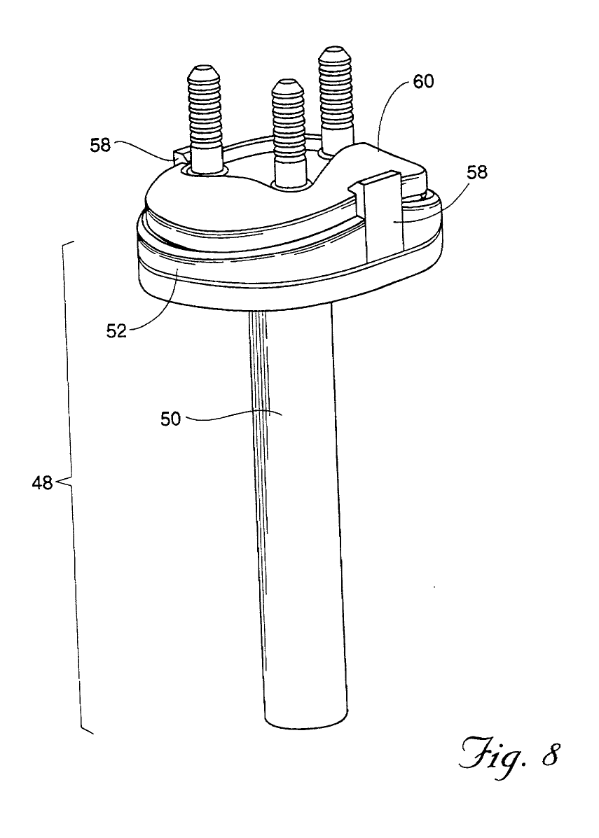

For the pins 43 to properly engage the openings 37, sufficient leverage must be applied to the cap 41 when seating the cap 41 onto the base 23. Toward this end, an insertion tool 48 is desirably provided, as shown in FIGS. 7 and 8.

The insertion tool 48 comprises a handle component 50 and a carrier component 52. The handle 50 is configured for grasping and of a length that assists in providing a desired angle and sufficient leverage for seating the cap 41.

As FIGS. 7 and 8 show, the carrier component 52 is adapted to mate with the cap 41 and can be variously configured. In the illustrated embodiment, the carrier 52 has a top surface 54 and a bottom surface 56. The top surface 54 is adapted to mate with the bearing surface 45 of the cap 41. In the illustrated embodiment, a pair of clasps 58 extend from the carrier component 52 and serve to couple a mating surface 56 on the circumferential margin of the cap 41 in a snap-fit engagement. This arrangement allows the cap 41 to be selectively removable from the carrier 52.

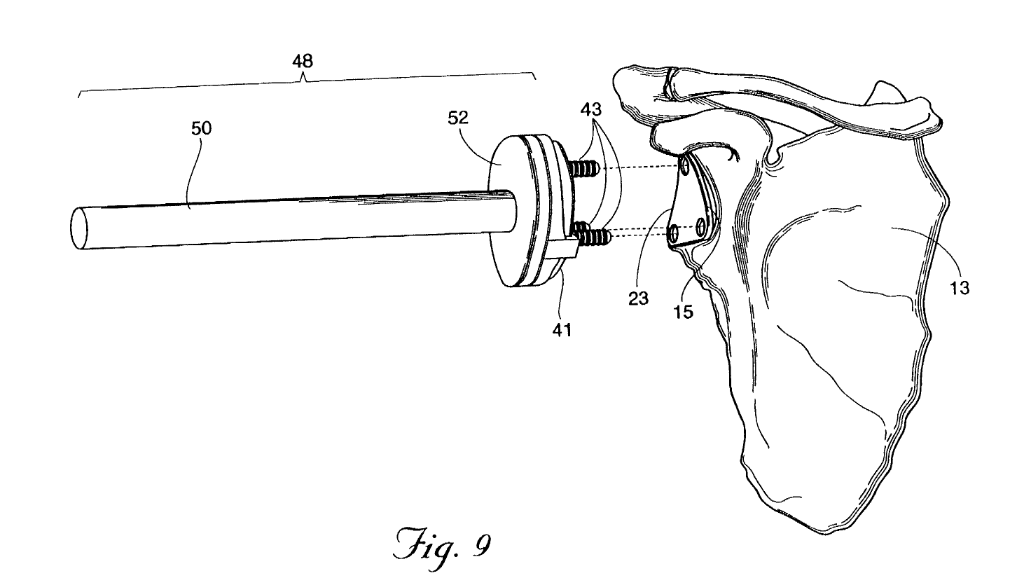

In use, as shown in FIG. 9, the base 23 is implanted into the glenoid cavity 15 of the scapula 13, as previously described. The cap 41 is coupled to the carrier 52 of the insertion tool 48.

The insertion tool 48 is then used to advance (shown by phantom lines in FIG. 9) the cap 41 into the glenoid cavity 15 and to place the cap 41 onto the base 23.

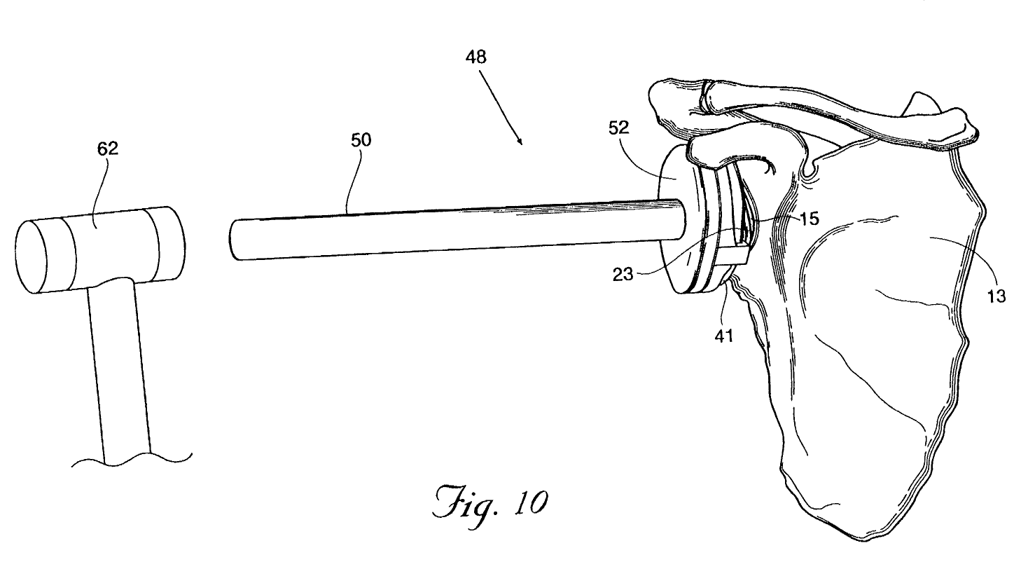

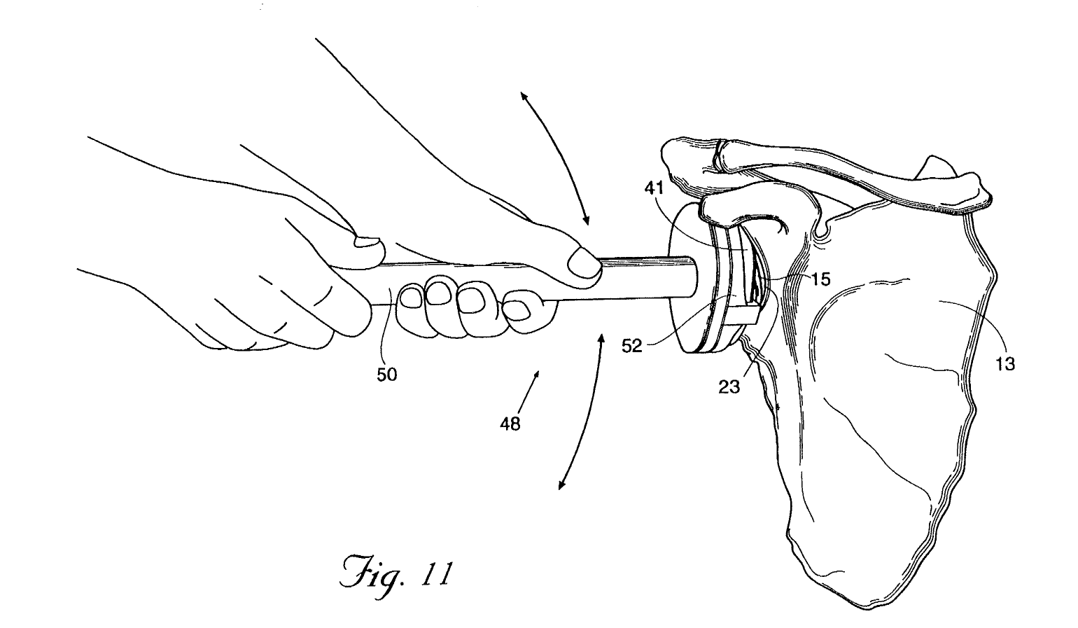

To mount and secure the cap 41 onto the base 23, force is exerted on the end of the handle 50, e.g., with a hammer 62, as shown in FIG. 10.

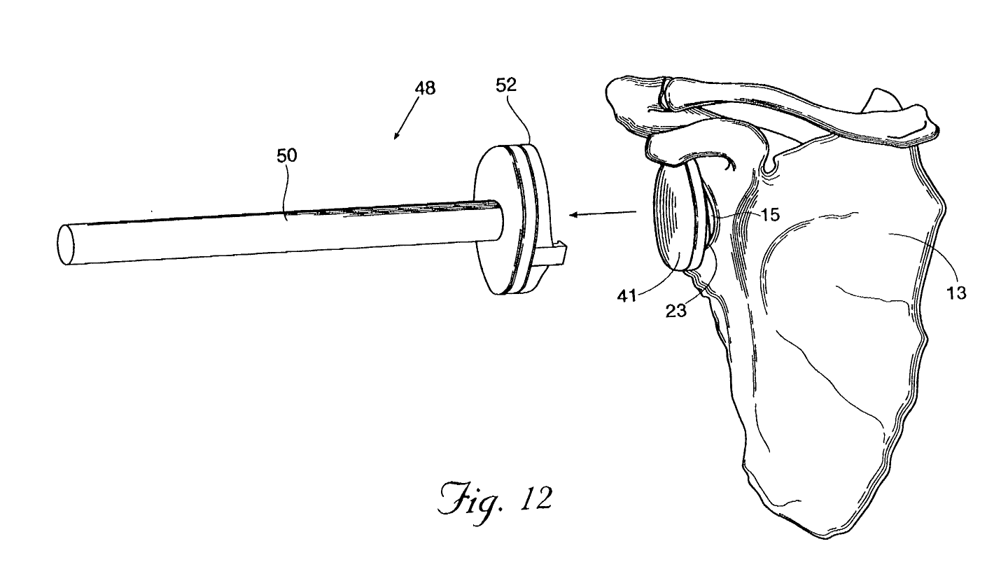

As represented by arrows in FIG. 11, the handle 50 is manipulated from side to side to release the cap 41 from the carrier 52. Finally, the insertion tool 48 is withdrawn, as FIG. 12 shows.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. A prosthetic implant for a bone structure, the implant comprising:
a base including at least one stem adapted to engage at least one void created in the bone structure, the stem having an exterior peripherally surrounding an interior lumen, and
a pin that fits into the interior lumen and expands the exterior of the stem.

2. An implant according to claim 1
wherein expansion of the stem compresses surrounding bone structure, to thereby secure the base to the bone structure.

3. An implant according to claim 1
wherein the base includes several stems, each having an exterior peripherally surrounding an interior lumen, and
wherein several pins are provided to fit into the interior lumens of the respective stems to expand the respective exteriors.

4. An implant according to claim 1
further including a cap from which the pin depends, wherein fitting the pin into the interior lumen of the stem couples the cap to the base.

5. An implant according to claim 4
wherein the cap includes a bearing surface, and
wherein the pin depends from the cap from a surface that faces away from the bearing surface.

6. An implant according to claim 4
wherein the cap and base include nesting surfaces that rest together when the base is coupled to the cap.

7. The method of mounting a prosthesis in a bone structure comprising the steps of:
providing a prosthetic implant that includes a base having at least one expandable stem having an exterior peripherally surrounding an interior lumen,
locating the implant on the bone structure by placing the stem into a void formed in the bone structure, and
securing the implant by inserting a pin in the interior lumen of the stem to expand the exterior of the stem within the surrounding bone structure.

8. A method according to claim 7
wherein the bone structure comprises a glenoid cavity of a shoulder joint.

9. A method according to claim 8,
wherein, prior to the securing step, the pin is releasably coupled to an insertion tool,
wherein, during the securing step, the insertion tool is manipulated to insert the pin into the stem, and
wherein, after the securing step, the pin is released from the insertion tool.

10. A prosthetic implant for a glenoid cavity of a shoulder joint comprising
a base including at least one stem adapted to engage at least one void created in bone structure in the glenoid cavity, the stem having an exterior peripherally surrounding an interior lumen, and
a pin that fits into the interior lumen to expand the exterior of the stem within surrounding bone structure to thereby secure the base to the bone structure.

11. An implant according to claim 10
further including a cap from which the pin depends, wherein fitting the pin into the interior lumen of the stem couples the cap to the base.

12. An implant according to claim 11
wherein the cap includes a bearing surface, and
wherein the pin depends from the cap from a surface that faces away from the bearing surface.

13. An insertion tool for use with a prosthetic implant for a bone structure as defined in claim 1 comprising
a handle, and
a carrier coupled to the handle and configured to hold the pin for insertion into the stem.

14. An insertion tool according to claim 13
wherein the carrier is configured to release the pin in response to manipulation of the handle.
